(19) United States
(12) Patent Application Publication
(10) Pub. No.: US 2017/0252221 A1
(43) Pub. Date: Sep. 7, 2017

(54) Adhesive Bandage With Integral Sticker

(71) Applicant: Kim Scopelliti, Elysburg, PA (US)

(72) Inventor: Kim Scopelliti, Elysburg, PA (US)

(21) Appl. No.: 15/053,348

(22) Filed: Feb. 25, 2016

(51) Int. Cl. A61F 13/02; A61F 13/00

(52) U.S. Cl. CPC A61F 13/0226; A61F 13/00059; A61F 13/0206; A61F 13/0259; A61F 2013/00153

(57) ABSTRACT

An adhesive bandage having at least one shape cut into the surface to produce a standalone sticker using the existing materials of the bandage. In one embodiment, a shape is kiss cut into the surface and adhesive layer of the bandage. When the release tabs are removed, the sticker remains on the release tab, leaving behind a decorative shape in the bandage. The user then may remove the sticker from the release tab and place it wherever desired. In another embodiment, the sticker can be die cut through all the bandage layers thus allowing use of the sticker before or after the bandage is applied.

### CROSS-REFERENCE TO RELATED APPLICATIONS

(0001) The present application claims the benefit of PPA Ser. Nr. 62/121,109 filed Feb. 26. 2015 by the present inventor, which is incorporated by reference.

### BACKGROUND-PRIOR ART

Adhesive bandages come in a variety of shapes, sizes, colors and designs. They are used to cover and protect wounds such as cuts, abrasions, punctures, and other forms of wounds inflicted on human tissue. Typically, an adhesive bandage comprises a flexible backing with an adhesive surface for attaching the adhesive bandage to the skin. Most, but not all, of these bandages have a patch of absorbent material disposed at the center of the adhesive surface. Such adhesive bandages are well known and staple items in a first aid kit or medicine cabinet.

Manufacturers have attempted to make bandages more attractive and entertaining, particularly to children, by the addition of colorful designs, imprinted shapes, designs and licensed movie and TV characters popular with children. Other bandages are manufactured in fun and novel shapes such as hearts and bacon. Stillothers have included decorations like stickers, tattoos or gems which are adhered to the bandage by the wearer.

It is known in the prior art for bandages with add on decorative or informational enhancements as disclosed in the following patents:

U.S. Pat. No. 4,094,316 issued to Nathanson on Jun 13, 1978 for an ADHESIVE BANDAGE WITH REUSABLE APPLIQUE;

U.S. Pat. No. 6,455,752 B1 issued to Vesey on Sep. 24, 2002 for a DECORATIVE ADHESIVE BANDAGE KIT;

U.S. Patent Application Publication No. 2005/0101899 A1 (Wegmann) on May 12, 2005 for a BAND AID APPLIQUE;

U.S. Patent Application Publication No. 2013/0046261 A1 (Reif) on Feb. 21, 2013 for a DECORATIVE ADHESIVE WOUND CARE SYSTEMS.

U.S. Patent No. 7,205,449 B2 (Levin) on Apr. 17, 2007 for a Self-Adhesive Bandage Indicating Wound Care Instructions.

U.S. Pat. No. 6,586,651 B2 issued to Sullivan on Jul. 1, 2003 for an ADHESIVE BANDAGE WITH SOFT, THREE-DIMENSIONAL TOY.

These prior devices provide some entertainment and decoration through applique as in U.S. Pat. No. 4,094,316 and U.S. Patent Application No. 2005/0101899 A1 and stickers supplied to apply to the bandage as in U.S. Pat. No. 6,455,752 B1 and U.S. Pat. No. 7,205,449 B2 and even a means to draw on the bandage as in U.S. Patent Application Publication No. 2013/0046261 A1. U.S Pat. No 6,586,651 B2, offers a three dimensional toy built into the adhesive bandage. While these adhesive bandages offer amusement or information, they have disadvantages, including:
a) To provide the entertainment or information additional materials must be supplied with the adhesive bandage.
b) Additional raw materials and resources must be used to create the entertainment within the adhesive bandage itself.
c) The amusement occurs on the bandage itself, encouraging the wearer to focus on and touch the wound site.

### SUMMARY OF EMBODIMENTS

A first embodiment includes:
a) an adhesive bandage comprising a backing, an adhesive layer on one side thereof and an absorbent pad affixed to the adhesive and at least one release tab affixed to the adhesive.
b) At least one shape kiss-cut into the bandage to form a stand-alone sticker remaining on the release tab when removed from the bandage. When the release tab with sticker affixed is removed from the bandage a decorative cut out shape is left in the adhesive bandage. A user may remove the sticker from the release tab and place it anywhere desired.

A second embodiment includes:
a) an adhesive bandage comprising a backing, an adhesive layer on one side thereof and an absorbent pad affixed to the adhesive and at least one release tab affixed to the adhesive.
b) At least one shape die-cut into the bandage to form a stand-alone sticker when removed from the bandage. When the sticker is removed from the bandage a decorative cut out shape is left in the adhesive bandage. A user may remove the sticker from the attached die-cut release tab and place it anywhere desired.

### Advantages:

Accordingly, several advantages of one or more aspects are as follows: to provide amusement in an adhesive bandage without requiring any additional material resources, that redirects the amusement from the wound site so the wound can be tended and protected, to provide a unique bandage look when the sticker is removed, that offer a canvas for unique colors, cut outs and designs. Other advantages of one or more aspects will be apparent from a consideration of the drawings and ensuing descriptions.

There has thus been outlined, rather broadly, some of the features of the embodiments in order that the detailed description thereof may be better understood, and in order that the present contribution to the art may be better appreciated. There are additional features of the embodiment that will be described hereinafter.

In this respect, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

Other advantages of the present embodiment will become obvious to the reader and it is intended that these advantages are within the scope of the present embodiments. To the accomplishment of the above and related objects, these may be embodied in the form illustrated in the accompanying drawings, attention being called to the fact, however, that the drawings are illustrative only, and that changes may be made in the specific construction illustrated and described within the scope of this application.

### BRIEF DESCRIPTION OF DRAWINGS

FIG. 1 schematically includes various other objects, features and attendant advantages of the present embodiment that will become fully appreciated as the same becomes better understood when considered in conjunction with the accompanying drawings, in which like reference characters designate the same or similar parts throughout the several views, and wherein:

FIG. 1 is an upper perspective view of the first embodiment of an adhesive bandage with integral sticker.

FIG. 2 is a side view of the first embodiment along cross section line 2 of the adhesive bandage with integral sticker.

FIG. 3 is a bottom view of the first embodiment of the adhesive bandage with integral sticker with one release tab removed.

FIG. 4 is an upper perspective view of a second embodiment of an adhesive bandage with integral sticker.

FIG. 5 is a side view of a second embodiment along cross section line 5 of the adhesive bandage with integral sticker.

FIG. 6 is a bottom view of a second embodiment of the adhesive bandage with integral sticker shown in FIG. 4 with one die-cut sticker removed.

FIG. 7 is a perspective view of a person using the adhesive bandage with integral stickers.

### DRAWINGS—REFERENCE NUMERALS

10 Adhesive Bandage

11 Backing Strip

12 Backing Strip Outer Surface

13 Backing Strip Inner Surface

15 Absorbent Pad

16 Absorbent Pad Upper Surface

17 Absorbent Pad Lower Surface

18a, b Release Tabs

20 Sticker

30 Decorative Void

### DETAILED DESCRIPTION OF SPECIFIC EMBODIMENTS

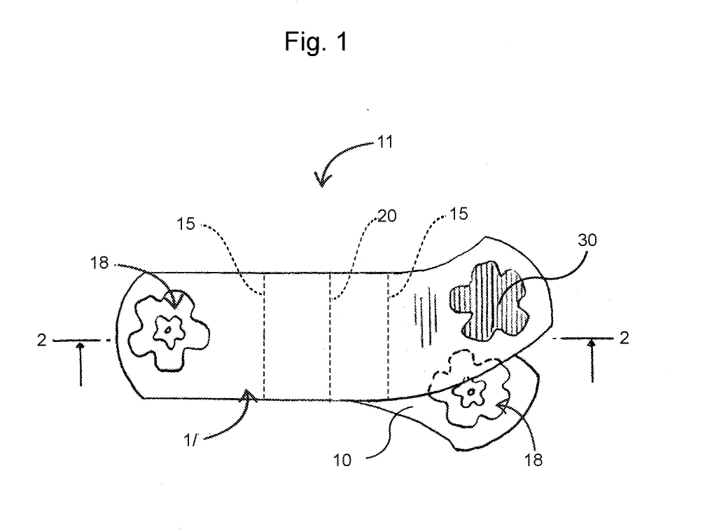

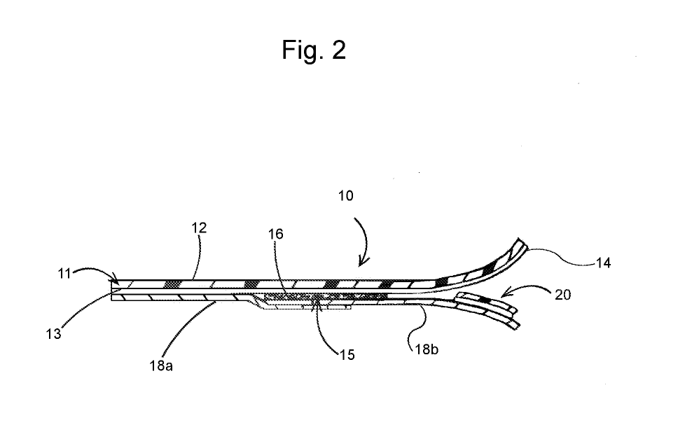

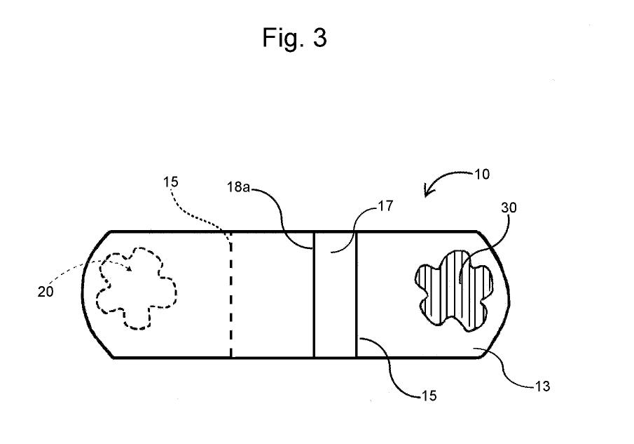

### Detailed Description—FIGS. 1-3, 7—First Embodiment

A bandage which may be used in the first embodiment is shown in FIGS. 1-3, 7 of the accompanying drawings. Adhesive Bandage 10 comprises a backing strip 11, a layer of adhesive 14, an absorbent, wound contacting pad 15, a pair of protective release tabs 18a and 18b, and at least one kiss-cut shape in the backing strip 11 and adhesive layer 14 which forms a sticker 20. When release tab 18a or 18b with the sticker 20 attached is removed, a decorative void 30 is left in the bandage 10.

As illustrated, the backing strip 11 is rectangular in configuration and comprises a first major, or outer surface 12 and a second major surface, or inner surface 13. The adhesive 14, which may be any of the well-known adhesive materials used for adhesive bandages, is applied to the inner surface 13 of backing strip 11.

The bandages may be of any size or shape where there is sufficient room to incorporate stickers or cutouts without affecting the ability of the adhesive bandage to stick to the skin of the wearer.

The upper surface 16 of the absorbent pad 15 is secured to the inner surface 13 of the backing strip 11 via the adhesive layer 14. The absorbent pad 15 is typically a non-woven material made from rayon fibers or polyester fibers or a blend of such fibers. It will be apparent to those skilled in the art that other fibers, for example, cotton fibers or polyolefin fibers may be used in constructing absorbent pad 15.

As seen in the drawings, the absorbent pad 15 is centered from end-to-end of the bandage 10 and its side edges are co-extensive with the longitudinally extending edges of backing strip 11. The bandage 10 may assume other configurations known in the art.

The Bandage 10 also comprises the pair of adhesive protecting release tabs 18a, 18b which may be made from paper coated with a silicone release material. It will be understood that the release coated surface of tabs 18a, 18b will contact the adhesive 14 and the release coating on release tabs 18a, 18b will be such that the adhesive 14 will remain on the bandage backing strip inner surface 13 rather than on the release tabs 18a, 18b when the tabs are removed to expose the adhesive 14 prior to application of the bandage 10 to the skin or the sticker 20 to the skin or any other surface.

The backing Strip 11 may assume any of a number of other configurations; for example, the backing strip 11 may be square or circular. The outer surface of the backing strip 12, may have a printed design to make the stickers 20 more interesting, however, it is not imperative.

The adhesive bandage 10 may have an island construction, for example, absorbent pad 15 may be located on the inner surface of backing strip 13 such that its end and side edges are spaced inwardly from the adjacent end and side edges of the backing strip 11.

Alternatively, the release tabs 18a, 18b may be made of a low surface energy plastic film such as polyethylene or polystyrene which, if desired, may have a silicone release material or the like applied thereto.

The sticker 20 is created from the adhesive bandage 10 by kiss cutting a shape into the bandage 10. When the release tab 18a, 18b is removed the sticker 20 comprising the backing strip 11 surface with the adhesive layer 14 attached to the release tab 18a, 18b, creating the sticker 20 which the user may remove from the release tab 18a, 18b and place anywhere. The function of the sticker 20 is for amusement, to relay information or for advertising.

The sticker 20 comprises all layers of the adhesive bandage 10, except the absorbent pad 15. The stickers 20 cannot be cut from the area where the absorbent pad 15 is located, as the adhesive layer 14 needs to come in contact with the surface in which the user wishes to adhere the sticker 20. Said sticker 20 is shaped through kiss-cutting technology.

The sticker 20 may be of any shape or size as long as it does not interfere with the main purpose of the bandage, to cover and protect a wound. The adhesive bandage 10 may have a single sticker 20 or a multitude of the stickers 20. The sticker 20 may even extend to the edge of the adhesive bandage 10 in some designs.

The sticker 20 may be die-cut with perforations, serrations, kiss-cut, laser cut, or produced in any of the well-known methods for producing cut-outs.

The sticker 20 does not even need to be included with the adhesive bandage 10 if the preference is simply to create a unique look to the bandage using die-cutting or laser-cutting technology.

When the sticker 20 is removed from the adhesive bandage 10, the decorative void 30 in the shape of the sticker 20 is left in the adhesive bandage 10. This void creates a decorative element in the adhesive bandage 10 providing a unique and novel look. The function of the decorative void 30 is for decoration, amusement, advertising and/or to relay information.

The decorative void 30 is the void left in the adhesive bandage 10 when the sticker 20 is removed for use elsewhere.

The decorative void 30 may be any size or shape as long as it does not interfere with the main purpose of the adhesive bandage 10, to cover and protect a wound. The cut out shape 30 may be singular or there may be a plurality of decorative voids 30 in the bandage 10. The decorative void 30 may even extend to the edge of the adhesive bandage 10.

### Operation—First Embodiment

A thin layer of the adhesive 14 is applied to the inner surface of backing strip 13. The absorbent pad 15 is affixed to the lower surface of the backing strip 12 through the use of the aforementioned adhesive 14. As seen in the drawings, the absorbent pad 15 is centered from end-to-end of the bandage and its side edges are co-extensive with the longitudinally extending edges of the backing strip 11.

The release coated surface of tabs 18a, 18b will contact the adhesive 14 and the release coating on the release tabs 18a, 18b will be such that the adhesive 14 will remain on the bandage backing strip 11 rather than on the release tabs 18a, 18b when the tabs are removed to expose the adhesive 14 prior to application of the bandage 10 to the skin or the sticker 20 to the skin or any other surface.

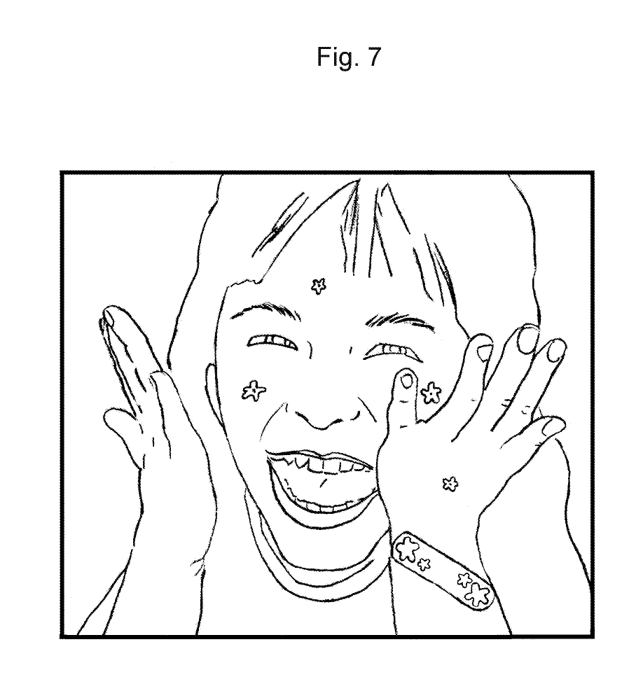

An end user simply removes the release tabs 18a, 18b from the adhesive bandage 10. When the release tabs 18a, 18b are removed, the kiss cut sticker 20 remains attached to the release tab with the adhesive 14. The user can then apply the bandage 10 with the decorative void 30 to the wound and peel the sticker 20 from the release tab 18a or 18b to adhere the sticker 20 to whatever surface they desire. An example of the embodiment in use can be seen in FIG. 7.

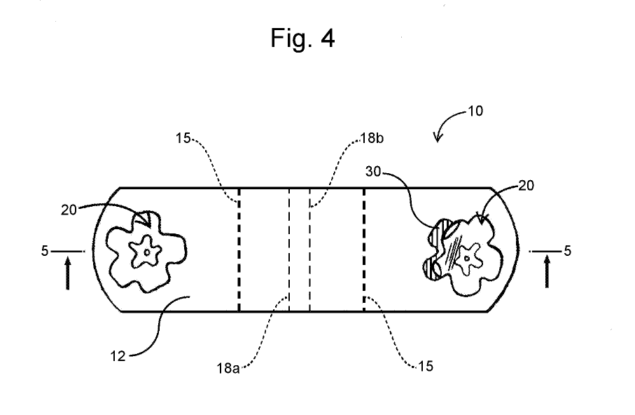

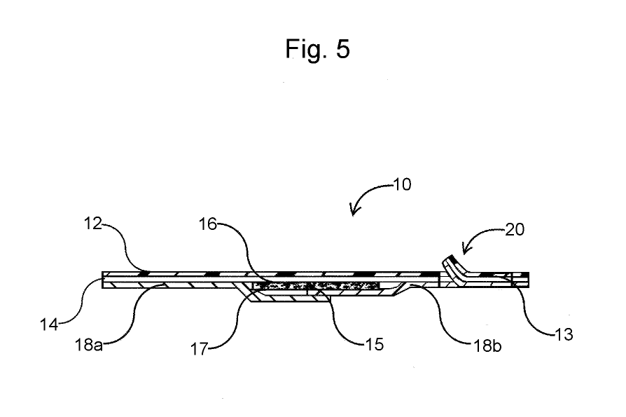

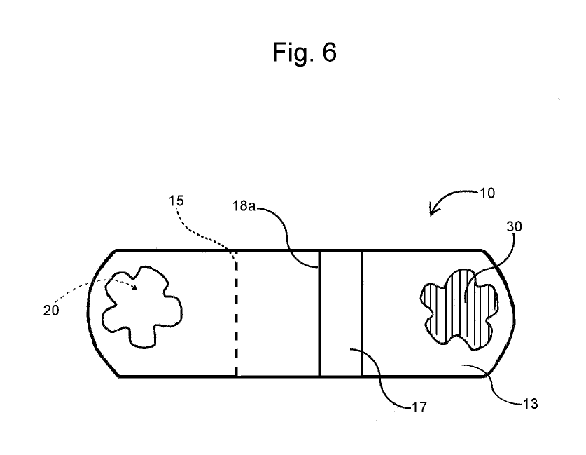

### Description—Second Embodiment—FIGS. 4-7

A bandage which may be used in the second embodiment is shown in FIGS. 4-7 of the accompanying drawings. Adhesive Bandage 10 comprises a backing strip 11, a layer of adhesive 14, an absorbent, wound contacting pad 15, a pair of protective release tabs 18a and 18b, and at least one die-cut shape in the backing strip 11, adhesive layer 14 and the release tab 18a and/or 18b which forms a sticker 20. When said sticker 20 is removed, a decorative void 30 is left in the bandage 10.

As illustrated, the backing strip 11 is rectangular in configuration and comprises a first major, or outer surface 12 and a second major surface, or inner surface 13. The adhesive 14, which may be any of the well-known adhesive materials used for adhesive bandages, is applied to said inner surface 13 of the backing strip 11.

The bandages may be of any size or shape where there is sufficient room to incorporate stickers or cutouts without affecting the ability of the adhesive bandage to stick to the wearer's skin.

The upper surface 16 of the absorbent pad 15 is secured to the inner surface 13 of the backing strip 11 via the adhesive layer 14. The absorbent pad 15 is typically a non-woven material made from rayon fibers or polyester fibers or a blend of such fibers. It will be apparent to those skilled in the art that other fibers, for example, cotton fibers or polyolefin fibers may be used in constructing the absorbent pad 15.

As seen in the drawings, the absorbent pad 15 is centered from end-to-end of the bandage 10 and its side edges are co-extensive with the longitudinally extending edges of said backing strip 11. The bandage 10 may assume other configurations known in the art.

The Bandage 10 also comprises the pair of adhesive protecting release tabs 18a, 18b which may be made from paper coated with a silicone release material. It will be understood that the release coated surface of tabs 18a, 18b will contact the adhesive 14 and the release coating on said release tabs 18a, 18b will be such that the adhesive 14 will remain on the bandage backing strip inner surface 13 rather than on the release tabs 18a, 18b when the tabs are removed to expose the adhesive 14 prior to application of the bandage 10 to the skin or the sticker 20 to the skin or any other surface.

The backing Strip 11 may assume any of a number of other configurations; for example, the backing strip 11 may be square or circular. The outer surface of the backing strip 12, may have a printed design to make the stickers 20 more interesting, however, it is not imperative.

The adhesive bandage 10 may have an island construction, for example, absorbent pad 15 may be located on the inner surface of backing strip 13 such that its end and side edges are spaced inwardly from the adjacent end and side edges of the backing strip 11.

Alternatively, the release tabs 18a, 18b may be made of a low surface energy plastic film such as polyethylene or polystyrene which, if desired, may have a silicone release material or the like applied thereto.

The sticker 20 is created from the adhesive bandage 10 by die cutting a shape into the bandage 10. The user removes the sticker 20 from the adhesive bandage 10 by breaking the die cut perforation which holds the sticker 20 in place. The sticker 20 shape is cut through the backing strip 11, the adhesive layer 14 and the corresponding release tab creating the sticker 20. The user may remove the sticker 20 from the bandage 10 and place the sticker 20 where desired. The function of the sticker 20 is for amusement, to relay information or for advertising.

The sticker 20 comprises all layers of the adhesive bandage 10, except the absorbent pad 15. The stickers 20 cannot be cut from the area where the absorbent pad 15 is located, as the adhesive layer 14 needs to come in contact with the surface in which the user wishes to adhere the sticker 20. Sticker 20 is shaped through die-cutting technology.

The sticker 20 may be of any shape or size as long as it does not interfere with the main purpose of the bandage, to cover and protect a wound. The adhesive bandage 10 may have a single sticker 20 or a multitude of the stickers 20. The sticker 20 may even extend to the edge of the adhesive bandage 10 in some designs.

The sticker 20 may be die-cut with perforations, serrations, laser cut or produced in any of the well-known methods for producing cut-outs.

The sticker 20 does not even need to be included with the adhesive bandage 10 if the preference is simply to create a unique look to the bandage using die-cutting or laser-cutting technology.

When the sticker 20 is removed from the adhesive bandage 10, the decorative void 30 in the shape of the sticker 20 is left in the adhesive bandage 10. This void creates a decorative element in the adhesive bandage 10 providing a unique and novel look. The function of the decorative void 30 is for decoration, amusement, advertising and/or to relay information.

The decorative void 30 is the void left in the adhesive bandage 10 when the sticker 20 is removed for use elsewhere.

The decorative void 30 may be any size or shape as long as it does not interfere with the main purpose of the adhesive bandage 10, to cover and protect a wound. The cut out shape 30 may be singular or there may be a plurality of decorative voids 30 in the bandage 10. The decorative void 30 may even extend to the edge of the adhesive bandage 10. An example of the embodiment in use can be seen in FIG. 7.

### Operation—Second Embodiment

A thin layer of the adhesive 14 is applied to the inner surface of backing strip 13. The absorbent pad 15 is affixed to the lower surface of the backing strip 12 through the use of adhesive 14. As seen in the drawings, the absorbent pad 15 is centered from end-to-end of the bandage and its side edges are co-extensive with the longitudinally extending edges of the backing strip 11.

The release coated surface of tabs 18a, 18b will contact the adhesive 14 and the release coating on the release tabs 18a, 18b will be such that the adhesive 14 will remain on the bandage backing strip 11 rather than on the release tabs 18a, 18b when said tabs are removed to expose the adhesive 14 prior to application of the bandage 10 to the skin or the sticker 20 to the skin or any other surface.

The sticker 20 remains attached to the adhesive bandage 10 via die cut perforations until user wishes to remove it. When the sticker is removed, the portion of the release tab 18a or 18b that was included in the die-cut shape becomes the sticker backing, protecting the adhesive layer 14 from affixing to a surface until the user is ready.

### Conclusion, Ramification and Scope

Accordingly, the reader will see that the adhesive bandage with integral sticker can provide amusement away from the wound site allowing the wound to be tended without the use of additional materials. Furthermore, the adhesive bandage with integral stickers has the additional advantages in that:
The bandages can be produced in a multitude of designs and colors to amuse and inform the user.
The bandages and integral stickers can be produced in a multitude of shapes to amuse and inform the wearer.
Games may be incorporated into the design. For example, a tic-tac-toe grid and x and o stickers may be imprinted on the adhesive bandage.

Although the description above contains many specificities, these should not be construed as limiting the scope of the embodiments but as merely providing illustrations of some of their embodiments. For example, the sticker can be of any size, shape or color as long as it does not inhibit the bandages ability to adhere to the wearer; there can be one or a plurality of stickers, the bandage material may be of a plastic or cloth material, etc.

Thus the scope of the embodiments should be determined by the appended claims and their legal equivalents, rather than by examples given.

What is claimed is:

1. A bandage comprising:
a. A generally thin planar and flexible backing layer having an outer surface and an opposing inner surface.
b. A generally thin planar and flexible adhesive layer attached to and covering the inner surface of said backing layer.
c. A generally thin planar and flexible absorbent layer having an upper surface and an opposing lower surface, wherein said backing layer laterally expands beyond at least two edges of said absorbent layer, and further wherein the upper surface of said absorbent layer is attached to the adhesive layer.
d. At least one generally thin planar and flexible release tab adhered to the adhesive layer to cover and protect said absorbent layer and said adhesive layer.
e. Wherein there is at least one shape cut into the portion of said backing layer which expands beyond said absorbent layer comprised of said backing layer and said adhesive layer, whereby a sticker is formed.

2. A bandage according to claim 1 wherein said sticker is cut through said backing layer, said adhesive layer and said release tab, whereby forming an independent sticker.

3. A bandage according to claim 1 that has pre-printed images, designs or colors.

4. A bandage according to claim 1 that includes additional decorative elements to enhance said stickers.

5. A bandage according to claim 1 that includes a means to affix said sticker(s) to such as, but not limited to, a preprinted paper or booklet.

6. A bandage according to claim 1 wherein said sticker expands to the edge of the bandage.

7. A bandage according to claim 2 wherein said sticker is cut through said backing layer, said adhesive layer and said release tab.

8. A bandage according to claim 2 that has pre-printed images, designs or colors.

9. A bandage according to claim 2 that includes additional decorative elements to enhance said stickers.

10. A bandage according to claim 2 that includes a means to affix said sticker(s) to such as, but not limited to, a preprinted paper or booklet.

11. A bandage according to claim 2 wherein said sticker expands to the edge of the bandage.
